(19) United States
(12) Patent Application Publication
(10) Pub. No.: US 2022/0273169 A1
(43) Pub. Date: Sep. 1, 2022

(54) SINGLE-PORT SPINAL SURGERY INSTRUMENT SET AND METHOD FOR USING SINGLE-PORT SPINAL SURGERY INSTRUMENT SET

(71) Applicant: Sang Ho LEE, Seoul (KR)

(72) Inventors: Sang Ho LEE, Seoul (KR); Chan Hong PARK, Daegu (KR)

(21) Appl. No.: 17/627,312

(22) PCT Filed: Jul. 14, 2020
(86) PCT No.: PCT/KR2020/009235

§ 371 Date: Jan. 14, 2022

(30) Foreign Application Priority Data
Jul. 15, 2019 (KR) 10-2019-0084886

(51) Int. Cl. A61B 1/313; A61B 17/29; A61B 17/34; A61B 90/00; A61B 17/32; A61B 18/22

(52) U.S. Cl. CPC A61B 1/3135; A61B 17/29; A61B 17/3417; A61B 90/361; A61B 17/320068; A61B 18/22; A61B 2217/007; A61B 2217/005

(57) ABSTRACT

The present invention relates to a surgery instrument set for single port spinal surgery, in which since a surgical instrument is inserted into an endoscope so as to secure a surgery space through a single port to perform surgery, an invasion may be minimized and a surgical instrument may be replaced with instruments having a variety of functions and shapes according to the purpose of surgery so as to reduce time consumed for the surgery. To this end, the present invention relates to a punch configured to form a treatment hole, an intubator inserted into the formed treatment hole and configured to secure a path, an endoscope inserted into the intubator and configured to secure a view of a surgery region, and a surgical instrument set including surgical instruments which are each replaceable with an adequate instrument according to the purpose and target of surgery and inserted into the endoscope to perform surgery and a method of using the same.

### TECHNICAL FIELD

The present invention relates to a surgical instrument set for spinal surgery, and more particularly, to a surgical instrument set for single port spinal surgery which is joined with an endoscope and secures a surgery space through a single port to minimize invasion and replaces a surgical instrument with instruments having a variety of functions and shapes according to the purpose of surgery to reduce time consumed for surgery and a method of using the same.

### BACKGROUND ART

Generally, spinal stenosis is a disease in which the spinal canal that is a path through which the spinal cord passes narrows due to an influence of anaplastia or the like and presses the spinal cord, and thus causes a variety of neurological indispositions such as pseudoclaudication of the cauda equina or nerve roots and the like. Central spinal stenosis that is a type of spinal stenosis occurs because thickened facet joints in a disk region invade the spinal canal, a height of the disk is lowered, a vertical length of the spinal canal is reduced, and thus soft tissue such as a yellow ligament, a facet joint membrane, a longitudinal ligament, or the like is thickened or ossified and occupies a considerable part of a lateral cross section of the spinal canal. To treat the corresponding disease, a laminectomy, a partial laminectomy, and the like which are surgeries for physically widening the stenosed spinal canal are performed.

Generally, in such surgical removal, a lamina of vertebral arch, a yellow ligament, and facet joints may be surgically removed or partially removed to remove factors oppressing a nerve element, and general decompression and synosteotomy including instrumentation and osteoplasty may be performed to prevent segmentation instability which may occur due to the removal. However, when this is performed, there are problems such as a delay of a surgery time, an increase in blood loss of a patient, premature anaplastia of vertical segmentation of an agglutination region, and the like. It is a current tendency to utilize a minimally invasive technique to remedy these problems.

Decompression utilizing a minimally invasive technique is a surgery method of removing a lesion area such as a thickened yellow ligament by inserting an endoscope and a surgical instrument through a minimum skin incision (2 mm to 5 mm), which is performed with minimal invasion, instead of directly opening the lesion area and has advantages such as less nerve synechia caused by a postoperative injury, the possibility to perform the surgery with local anesthesia with no risk of paralysis, little sequela, and a short recovery time.

As an example of utilizing the minimally invasive technique, Korean Patent Registration No. 10-1441320 discloses a surgical instrument set for performing minimally invasive endoscopic spinal surgery. However, since a publicly known conventional decompression treatment utilizing minimal invasion is generally performed by inserting an endoscope and a surgical instrument into different ports and uses two ports, an invaded area increases and there is a risk of an occurrence of unnecessary damage inside the human body due to the endoscope inserted to observe the surgical site.

### RELATED ART DOCUMENTS

### Patent Documents

Korean Patent Registration No. 10-1441320 (registered on Sep. 11, 2014)
Korean Patent Publication No. 10-2017-7011813 (published on Jul. 4, 2017)

### DISCLOSURE

### Technical Problem

The present invention is directed to providing a surgical instrument set for single port spinal surgery to allow surgery to be performed with minimum invasion through a single port using one surgical instrument without using an additional path in addition to a path through which an endoscope is inserted.

The present invention is also directed to providing a surgical instrument set for single port spinal surgery in which an endoscope and a surgical instrument are inserted in a joined form, thus securing a view and a surgery space.

The present invention is directed to providing a surgical instrument set for single port spinal surgery which is capable of more efficiently performing surgery by replacing a surgical instrument joined with an endoscope with a variety of surgical instruments such as a forceps, an ultrasonic instrument, a laser instrument, and the like according to the purpose of surgery.

Technical aspects of the present invention are not limited to the above-stated exemplary technical aspects, and other unstated technical aspects will be apparent to those of ordinary skill in the art from the following description.

### Technical Solution

One aspect of the present invention provides a punch, an intubator, an endoscope that can be inserted into the intubator, and a surgical instrument inserted into the endoscope.

The surgical instrument may be replaced with a variety of surgical instruments such as a forceps, an ultrasonic instrument, a laser instrument, and the like.

### Advantageous Effects

According to the present invention, since it is possible to perform surgery while approaching a surgery target part through only one path without unnecessary incision, there is provided an effect of minimizing bodily injury to a patient.

According to the present invention, since bodily injury to a patient is minimized, there is an effect of minimizing sequela and also reducing a recovery period.

Also, since it is possible to perform surgery while capturing images of a surgery target part using only one secured path, there is an effect of allowing a practitioner to maximally demonstrate his or her ability.

Also, since it is possible to replace a surgical instrument with a surgical instrument appropriate for the purpose of surgery in one secured path, there is an effect of reducing a surgery time.

Ultimately, there are effects of reducing a recovery period of a patient by minimizing injury of an invaded part and of increasing efficiency of surgery by securing a surgery space while capturing images through only a single path.

### DESCRIPTION OF DRAWINGS

FIG. 1 is an exploded view illustrating components according to one embodiment of the present invention.

FIG. 2 is a perspective view illustrating a punch according to one embodiment of the present invention.

FIG. 3 is a perspective view illustrating an intubator according to one embodiment of the present invention.

FIG. 4 is a perspective view illustrating an endoscope according to one embodiment of the present invention.

FIG. 5 is a cross-sectional view illustrating a cross section of an insertion portion included in the endoscope according to one embodiment of the present invention.

FIG. 6 is a perspective view illustrating a forceps according to one embodiment of the present invention.

FIG. 7 is a perspective view illustrating a detailed structure of the forceps according to one embodiment of the present invention.

FIG. 8 is a perspective view illustrating pincers of the forceps according to one embodiment of the present invention.

FIG. 9 is a perspective view illustrating joining of the punch and the intubator according to one embodiment of the present invention.

FIG. 10 is a perspective view illustrating joining of the intubator and the endoscope according to one embodiment of the present invention.

FIG. 11 is a perspective view illustrating joining of the intubator, the endoscope, and the forceps according to one embodiment of the present invention.

FIG. 12 is a perspective view illustrating joining of the intubator, the endoscope, and an ultrasonic instrument according to one embodiment of the present invention.

FIG. 13 is a perspective view illustrating joining of the intubator, the endoscope, and a laser instrument according to one embodiment of the present invention.

FIG. 14 is a perspective view illustrating a use state of a surgical instrument set according to the present invention.

FIG. 15 is a flowchart illustrating an operation of joining the punch with the intubator according to one embodiment of the present invention.

FIG. 16 is a flowchart illustrating an operation of separating the punch from the intubator according to one embodiment of the present invention.

FIG. 17 is a flowchart illustrating an operation of joining the intubator with the endoscope according to one embodiment of the present invention.

FIG. 18 is a flowchart illustrating an operation of joining the intubator, the endoscope, and the forceps with one another according to one embodiment of the present invention.

### MODE FOR INVENTION

Hereinafter, embodiments of the present invention will be described in detail with reference to the attached drawings to be easily executed by one of ordinary skill in the art. However, the present invention can be implemented in a variety of different forms and is not limited to the embodiments disclosed herein. Also, throughout the drawings, to clearly disclose the present invention, parts irrelevant to the present invention will be omitted. In the drawings, like or similar reference numerals refer to like or similar components.

The purposes and effects of the present invention may be naturally understood or more apparent from the following description, and it should be noted that the purposes and effects of the present invention are not limited to the following specification. Also, in description of the embodiments of the present invention, detailed description of well-known arts related to the present invention will be omitted when it is deemed to unnecessarily obscure the essentials of the present invention.

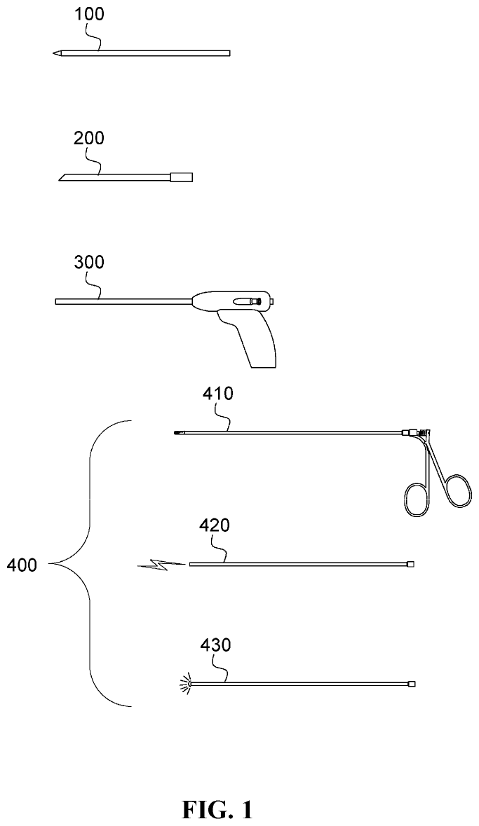

Hereinafter, embodiments of the present invention will be described in detail with reference to the attached drawings. Unless particularly defined otherwise in the specification, all “one ends” described hereafter will be on the left and “other ends” will be on the right on the basis of FIG. 1. Also, all “upper parts” described hereafter will be on an upper side and “lower parts” will be on a lower side on the basis of FIG. 1.

FIG. 1 is an exploded view illustrating components according to one embodiment of the present invention.

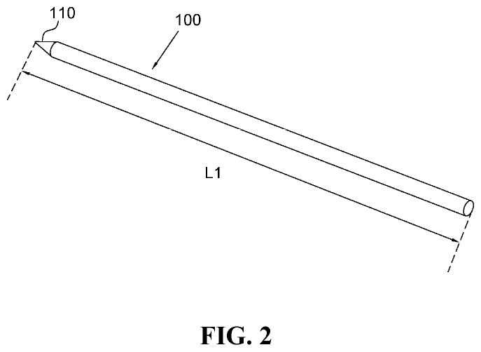

Referring to FIG. 1, a surgical instrument set for a single port spinal surgery includes a punch 100, an intubator 200, an endoscope 300, and a surgical instrument 400. In more detail, the surgical instrument 400 may be replaced, according to the goal of surgery, with a forceps 410, an ultrasonic instrument 420, and a laser instrument 430, and may be replaced with any other surgical instrument appropriate for the goal of surgery. The punch 100, the intubator 200, the endoscope 300, and the surgical instrument 400 will be described below in detail with reference to FIGS. 2 to 7.

FIG. 2 is a perspective view illustrating the punch 100 according to one embodiment of the present invention.

The punch 100 is an instrument configured to form a treatment hole H in the skin above a surgery target part T to allow the surgical instrument 400 to approach the surgery target part T.

In more detail, the punch 100 is an instrument configured to form a treatment hole H at a position at which a shortest distance from the skin to a thickened yellow ligament that is the surgery target part T can be secured or at a position from which a path for minimizing damage to internal organs can be secured.

Referring to FIG. 2, the punch 100 may have a long rod shape. A head portion 110 having a shape capable of easily entering the surgery target part T while forming the treatment hole H and of minimizing body injuries may be provided on the one end of the punch 100. In detail, the head portion 110 may be formed to be sharp enough to cut and enter the skin or may be formed to have a dull and blunt surface to minimize bodily injury. In more detail, the head portion 110 may have a conic shape having a width that gradually decreases toward the one end.

As an embodiment, the punch 100 may be implemented to have a first length L1 of 200 mm to 300 mm which is an entire length from a fore end to the other end of the punch 100 and to have a diameter of 5 mm to 6.75 mm. Here, most preferably, the first length L1 may be 240 mm to be most adequate for approaching the thickened yellow ligament that is the surgery target part T from the treatment hole H and the diameter may be 5.75 mm to minimize a size of the treatment hole H, but they are not limited thereto and may be provided depending on a position of the surgery target part T and the degree of invasiveness.

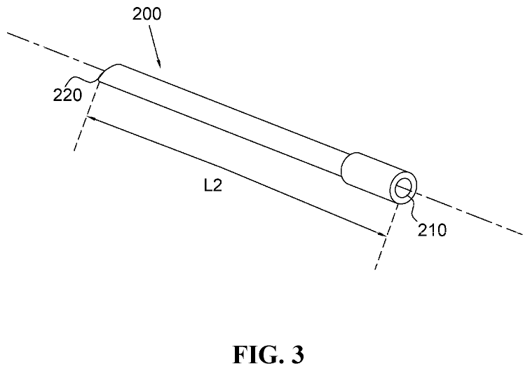

FIG. 3 is a perspective view illustrating the intubator 200 according to one embodiment of the present invention.

The intubator 200 is a device configured to secure a space (path) from the treatment hole H to the thickened yellow ligament that is the surgery target part T to allow the endoscope 300, the surgical instrument 400, and the like which will be described below in detail to enter. In detail, since the intubator 200 has a tubular shape and is inserted from the other end of the punch 100 along an outer circumferential surface of the punch 100, even when the punch 100 which has been inserted is removed, it is possible to secure a space for allowing the endoscope 300 and the surgical instrument 400 to enter. That is, the one end of the intubator 200 may be inserted into the treatment hole H to allow the one ends of the endoscope 300 and the surgical instrument 400 to be inserted into the other end of the intubator 200 having a tubular shape to secure the space for allowing the endoscope 300 and the surgical instrument 400 to enter. Here, to prevent parts of the body or internal organs from being injured or to prevent the inside of the intubator 200 from being contaminated through a gap between joining portions of the intubator 200 and the punch 100, a distance between the outer circumferential surface of the punch 100 and an inner circumferential surface of the intubator 200 may be formed to be minimized. To this end, the intubator 200 may be implemented to have an outer diameter of 6 mm to 7 mm. In more detail, most preferably, the outer diameter of the intubator 200 may be 7 mm, but is not limited thereto and may differ according to an outer diameter of the punch 100.

Referring to FIG. 3, the intubator 200 has a long rod shape having an open tube thereinside, includes a fore end opening portion 210 provided on the other end and configured to allow the endoscope 300 which will be described below in detail to be inserted into the intubator 200, and includes a distal end opening portion 220 provided on the one end and configured to allow a distal end of the endoscope 300 to pass through and protrude from the tube formed inside the intubator 200. In detail, an outer diameter of the fore end opening portion 210 provided on the other end of the intubator 200 may be formed to be greater than diameters of the intubator 200 and the distal end opening portion 220 to be distinguished from the distal end opening portion 220 provided on the one end of the intubator 200, and the distal end opening portion 220 provided on the one end of the intubator 200 may be formed so that a distal end has an inclined cross section to secure a path inside the body. In detail, one cross section of the intubator 200 may include an inclined surface with a certain angle to facilitate securing of a path into the body and entry into the body. In more detail, a second length L2 that is a length from a fore end to the distal end of the intubator 200 may be implemented to be 165 mm to 175 mm to facilitate entry into the body along the outer circumferential surface of the punch 100. Here, to facilitate removal of the punch 100 after the intubator 200 is inserted, most preferably, the second length L2 of the intubator 200 may be 170 mm, but is not limited thereto and may differ according to the first length L1 of the punch 100.

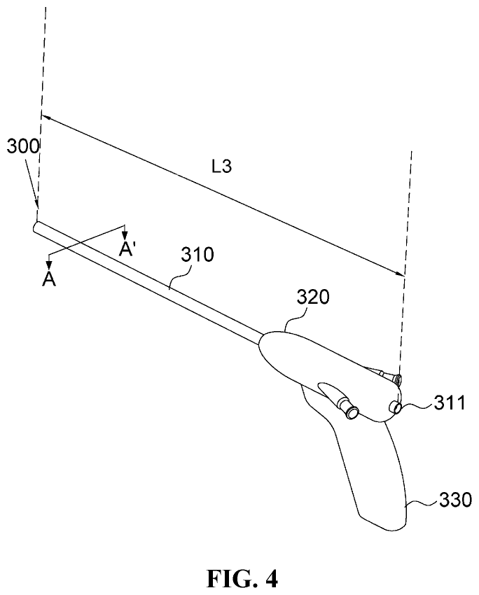

FIG. 4 is a perspective view illustrating the endoscope 300 according to one embodiment of the present invention.

The endoscope 300 is an instrument inserted into the intubator 200 to capture an image of the surgery target part T in real time and to provide a path to allow the surgical instrument 400 which will be described below in detail to reach the thickened yellow ligament that is the surgery target part T.

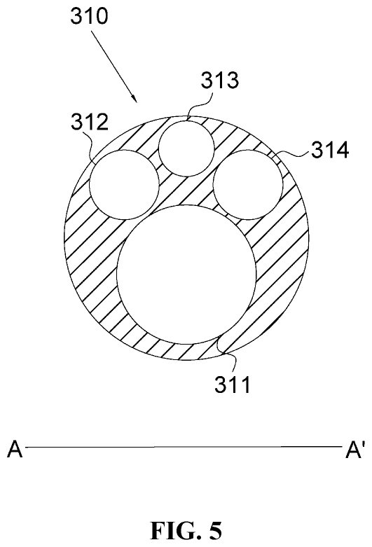

Referring to FIG. 4, the endoscope 300 includes an insertion portion 310 which is inserted into the intubator 200 through the fore end opening portion 210 provided on the other end of the intubator 200, and the insertion portion 310 includes a guide hole 311, an abstergent supply portion 312, an image-capturing portion 313, and an abstergent discharge portion 314 therein which will be described below in detail with reference to FIG. 5. In detail, an outer diameter of the insertion portion 310 may be formed to be smaller than an inner diameter of the intubator 200 to be inserted into the tube provided inside the intubator 200. In detail, the outer diameter of the insertion portion 310 may be formed to be 5.5 mm to 6.1 mm, and a third length L3 from a fore end to a distal end may be formed to be 260 mm to 270 mm. As a more exemplary embodiment, the insertion portion 310 has an outer diameter of 5.8 mm so that it can be easily inserted into the tube inside the intubator 200 and is formed so that the third length L3 is 264 mm to allow a practitioner to easily operate the endoscope 300, but is not limited thereto and may be variously provided according to a position of the surgery target part T.

Referring to FIG. 4, the endoscope 300 may include a body portion 320 on the other end of the insertion portion 310 to facilitate insertion of the surgical instrument 400. In detail, the body portion 320 may include tubes thereinside which are connected to the guide hole 311, the abstergent supply portion 312, and the abstergent discharge portion 314 which are provided inside the insertion portion 310. In more detail, the tubes provided inside the body portion 320 may be configured to have a tubular shape passing through a central part in the body portion 320 in which distal ends of the one end portions are connected to distal ends of other end portions of the guide hole 311, the abstergent supply portion 312, and the abstergent discharge portion 314 which are formed in the insertion portion 310 and parts of distal ends of the other end portions protrude from the other end of the body portion 320 to be distinguished from an outer surface of the body portion 320. This allows the practitioner to more easily inject an abstergent and insert the surgical instrument 400 into the endoscope 300.

Referring to FIG. 4, the endoscope 300 may include a handle portion 330 on a lower end of the body portion 320 so that it can be easily operated. In detail, when the practitioner sees a real-time data screen of the surgery target part T observed through the image-capturing portion 313 of the endoscope 300 and determines that it is necessary to move a position of the surgical instrument 400 which is inserted into the insertion portion 310 of the endoscope 300 to secure a surgery space while performing a surgery, the practitioner may hold the handle portion 330 with a hand which does not hold the surgical instrument 400 and may minutely change a direction to move a position of the surgical instrument 400 within a range in which blood vessels, muscles, nerves, and the like near the thickened yellow ligament that is the surgery target part T are not injured.

In more detail, the handle portion 330 may include an operation button configured to open or close paths of the abstergent supply portion 312 and the abstergent discharge portion 314 which are included in the insertion portion 310 or an operation button configured to allow the image-capturing portion 313 included in the insertion portion 310 to start, finish, temporarily stop, or record image-capturing to allow the practitioner to manipulate the endoscope 300 by hand without using the hand which is holding the surgical instrument 400.

FIG. 5 is a cross-sectional view illustrating a cross section of the insertion portion 310 included in the endoscope 300 according to one embodiment of the present invention.

The insertion portion 310 included in the endoscope 300 has a long rod shape and includes the guide hole 311 having an open tubular shape therein, the abstergent supply portion 312 configured to supply an abstergent to the surgery target part T using a nozzle or the like, the image-capturing portion 313 configured to capture images of the surgery target part T in real time, and the abstergent discharge portion 314 configured to suction an abstergent with which cleaning is finished and to discharge the abstergent to the outside of the endoscope 300.

In detail, the guide hole 311 provided inside the insertion portion 310 is formed to have an open tubular shape from a distal end of the other end to a distal end of the one end of the insertion portion 310 to allow the surgical instrument 400 which will be described below in detail to reach the thickened yellow ligament that is the surgery target part T. In more detail, the guide hole 311 and the abstergent discharge portion 314 which are provided inside the insertion portion 310 are formed not to intersect with each other so that the surgical instrument 400 inserted into the guide hole 311 inside the endoscope 300 and the abstergent which is contaminated after cleaning the surgery target part T may be prevented from coming into contact with each other and thus the surgery may be sanitarily performed.

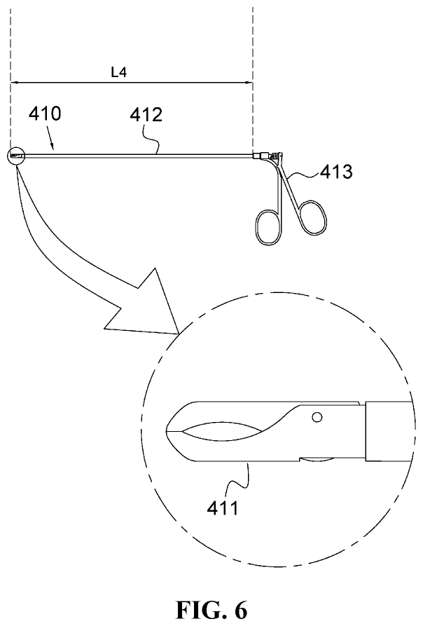

Hereinafter, the surgical instrument 400 that is one component of the present invention will be described in detail. The surgical instrument 400 that is one component of the present invention may be the forceps 410 which will be described below in detail with reference to FIG. 6 and may be replaced with the ultrasonic instrument 420, the laser instrument 430, or another surgical instrument appropriate for the purpose and target of surgery.

The ultrasonic instrument 420 that is one example of the surgical instrument 400 is an instrument configured to cut the thickened yellow ligament that is the surgery target part T using ultrasonic waves. In detail, the ultrasonic instrument 420 has a long rod shape and includes a device on the one end of the rod which is configured to generate ultrasonic waves to cut tissue using the device. In more detail, an outer diameter of the ultrasonic instrument 420 may be formed to be smaller than an inner diameter of the guide hole 311 of the endoscope 300.

The laser instrument 430 that is another example of the surgical instrument 400 is an instrument configured to suture cross sections of cut tissue using lasers after the thickened yellow ligament that is the surgery target part T has been cut. In detail, the laser instrument 430 has a long rod shape and includes a device on the one end of the rod which is configured to generate lasers to suture cross sections using the device. In more detail, an outer diameter of the laser instrument 430 may be formed to be smaller than the inner diameter of the guide hole 311 of the endoscope 300.

Since the structures, functions, and effects of the ultrasonic instrument 420 and the laser instrument 430 are well known to those skilled in the art, more detailed description thereof will be omitted.

Hereinafter, the forceps 410 will be described in detail with reference to FIGS. 6 to 8 as one example of the surgical instrument 400 that is one component of the present invention.

FIG. 6 is a perspective view illustrating the forceps 410 according to one embodiment of the present invention.

The forceps 410 that is one example of the surgical instrument 400 is an instrument inserted into the guide hole 311 provided inside the endoscope 300 to grip the thickened yellow ligament that is the surgery target part T.

Referring to FIG. 6, the forceps 410 includes a rod portion 412 inserted into the guide hole 311 provided inside the endoscope 300, a pincer portion 411 having pincer shapes provided on the one end of the rod portion 412, and a manipulation portion 413 having the shape of scissor handles and provided on the other end of the rod portion 412. In detail, outer diameters of the pincer portion 411 and the rod portion 412 of the forceps 410 are formed to be smaller than the inner diameter of the guide hole 311 of the endoscope 300, and the manipulation portion 413 of the forceps 410 is formed to have the shape of scissor handles to be moved forward or backward by manipulation of the practitioner to allow the pincer portion 411 to move like pincers.

In detail, the outer diameters of the pincer portion 411 and the rod portion 412 may be 2 mm to 3 mm, and a fourth length L4 from a distal end of a fore end portion of the pincer portion 411 to a distal end of the other end of the rod portion 412 may be 330 mm to 350 mm. As a more exemplary embodiment, the pincer portion 411 and the rod portion 412 may have outer diameters of 2.5 mm to be easily inserted into the guide hole 311 of the endoscope 300 and the fourth length L4 of the rod portion 412 may be 300 mm so that it can be easily manipulated by the practitioner, but they are not limited thereto and may have a variety of sizes according to the purpose of surgery.

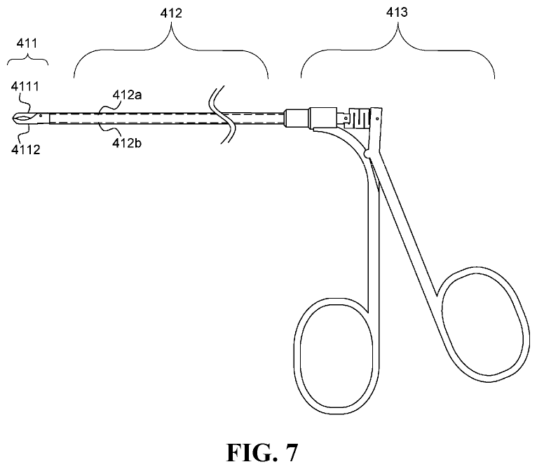

Hereinafter, a detailed structure of the forceps 410 will be described with reference to FIG. 7. FIG. 7 is a perspective view illustrating the detailed structure of the forceps 410 according to one embodiment of the present invention.

Referring to FIG. 7, the pincer portion 411 included in the forceps 410 includes a first pincer 4111 hinge-connected to the one end of the rod portion 412 and a second pincer 4112 fixed to the one end of the rod portion 412, the rod portion 412 included in the forceps 410 includes a transfer portion 412a having the one end connected to the other end of the first pincer 4111 and the other end connected to the one end of the manipulation portion 413 and a tube portion 412b having the one end connected to the other end of the second pincer 4112 and the other end connected to the one end of the manipulation portion 413. In detail, a force generated by forward or backward movement of the manipulation portion 413 of the forceps 410 according to manipulation of the practitioner may be transferred to the first pincer 4111 having the other end connected to the one end of the transfer portion 412a through the transfer portion 412a having the other end connected to the one end of the manipulation portion 413 to allow the first pincer 4111 to move upward or downward like pincers.

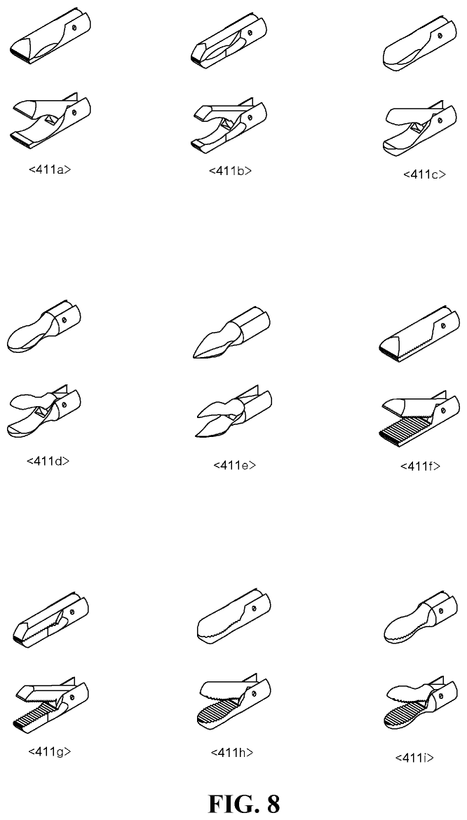

Hereinafter, a variety of embodiments of the forceps 410 will be described with reference to FIG. 8. FIG. 8 is a perspective view illustrating the pincer portion 411 of the forceps 410 according to one embodiment of the present invention.

Referring to FIG. 8, the pincer portion 411 of the forceps 410 may be implemented in a variety of forms according to a target to be gripped. In detail, the pincer portion 411 may be implemented in shapes with no groove inside the first pincer 4111 and the second pincer 4112 like <411-a> to <411-e> shown in FIG. 8 or may be implemented in shapes with certain patterns on the insides of the first pincer 4111 and the second pincer 4112 like <411-g> to <411-i> shown in FIG. 8. Here, the certain patterns are patterns such as zigzags, protrusions, and the like formed on cross sections to prevent a gripped target from being detached from the forceps.

Also, according to the target to be gripped, the pincer portion 411 may be implemented so that lengths, heights, and widths of overall shapes of the first pincer 4111 and the second pincer 4112 are uniform and the first pincer 4111 and the second pincer 4112 are cut to allow widths of ends thereof to be different like <411-c> shown in FIG. 8 and may be implemented so that the first pincer 4111 and the second pincer 4112 have concave intermediate parts and convex ends like <410-d> shown in FIG. 8.

As an exemplary embodiment, as in <411-a> of FIG. 8, the pincer portion 411 has pincer shapes and may be cut so that inner surfaces of the first pincer 4111 and the second pincer 4112 which spread in an upward direction and in a downward direction, respectively, are cut concavely, end parts of the first pincer 4111 and the second pincer 4112 are cut to be inclined, and the widths of the ends of the first pincer 4111 and the second pincer 4112 are cut to be uniform.

As another embodiment, like <411-b> shown in FIG. 8, the pincer portion 411 may be implemented so that the inner surfaces of the first pincer 4111 and the second pincer 4112 are concave, distal end portions of an upper side of the first pincer 4111 and a lower side of the second pincer 4112 are cut to be inclined, widths of ends of the first pincer 4111 and the second pincer 4112 are cut to be uniform, and both side surfaces of the first pincer 4111 and the second pincer 4112 are cut to allow the first pincer 4111 and the second pincer 4112 to have smaller widths than those of the embodiment shown in <411-a> of FIG. 8.

As still another embodiment, like <411-c> shown in FIG. 8, the pincer portion 411 may be cut so that the inner surfaces of the first pincer 4111 and the second pincer 4112 are concave, the distal end portions of the upper side of the first pincer 4111 and the lower side of the second pincer are not angularly cut and are trimmed to form a fluid curved shape overall when the first pincer 4111 and the second pincer 4112 are engaged with each other, and the first pincer 4111 and the second pincer 4112 are inclined to allow central widths of the ends to be longest.

As another embodiment, like <411-d> shown in FIG. 8, the pincer portion 411 may be cut so that the inner surfaces of the first pincer 4111 and the second pincer 4112 are concave, the upper side of the first pincer 4111 and the lower side of the second pincer 4112 are both trimmed to form concave centers and convex distal ends to allow a fore end portion of the pincer portion 411 to have a sphere shape in a side view, and the first pincer 4111 and the second pincer 4112 are inclined to allow central widths of the ends to be longest.

As another exemplary embodiment, like <411-e> shown in FIG. 8, the pincer portion 411 may be cut so that the widths of the first pincer 4111 and the second pincer 4112 gradually narrow toward ends thereof and the inner surfaces of the first pincer 4111 and the second pincer 4112 are both concave. In detail, the pincer portion 411 may easily catch a target to be gripped in the surgery target part T using the ends of the first pincer 4111 and the second pincer 4112 which are cut to be sharp. As another example of this, the second pincer 4112 may have a longer length than the first pincer 4111 to easily catch the target to be gripped.

In addition, like <411-h> to <411-i> shown in FIG. 8, the first pincer 4111 and the second pincer 4112 may be implemented to have flat inner surfaces having grooves.

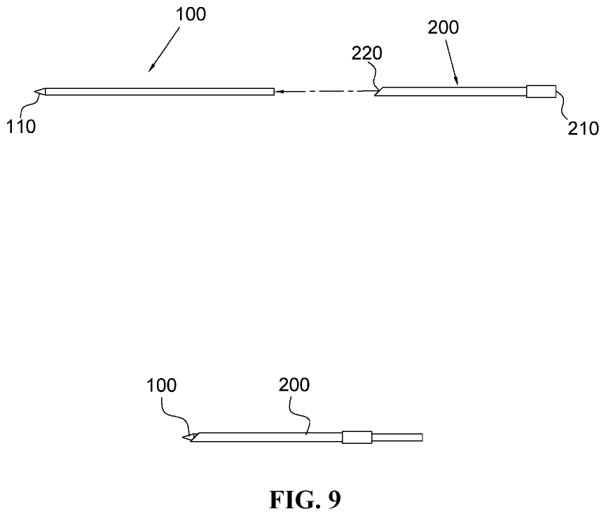

Hereinafter, joining relationships among the respective components of the present invention will be described with reference to FIGS. 9 to 13. Referring to FIGS. 9 to 13, the present invention provides joining relationships in which the intubator 200 is joined along the outer circumferential surface of the punch 100, the punch 100 is separated from the intubator 200, the endoscope 300 is joined with the inside of the intubator 200, and the surgical instrument 400 is joined with the inside of the endoscope 300. In detail, according to the purpose and target of surgery, the surgical instrument 400 may be joined to be replaceable with the forceps 410, the ultrasonic instrument 420, the laser instrument 430, or another appropriate surgical instrument.

Hereinafter, the joining relationships among the respective components of the present invention will be described in more detail with reference to FIGS. 9 to 13.

FIG. 9 is a perspective view illustrating joining of the punch 100 and the intubator 200 according to one embodiment of the present invention. The intubator 200 is joined along the outer circumferential surface of the punch 100.

Referring to FIG. 9, the intubator 200 is joined along the outer circumferential surface of the punch 100 so that the other end of the punch 100 which has formed the treatment hole H can be inserted into the distal end opening portion 220 provided on the one end of the intubator 200 and protrude through the fore end opening portion 210 of the intubator 200 and the head portion 110 of the punch 100 can protrude through the distal end opening portion 220 provided on the one end of the intubator 200.

In detail, the intubator 200 needs to include a length with which the fore end opening portion 210 provided on the other end can be on an outer surface of the treatment hole H while the distal end opening portion 220 provided on the one end of the intubator 200 approaches the surgery target part T, and the punch 100 needs to include a length with which the other end may protrude through the fore end opening portion 210 provided on the other end of the intubator 200 while the head portion 110 provided on the one end protrudes through the distal end opening portion 220 provided on the one end of the intubator 200.

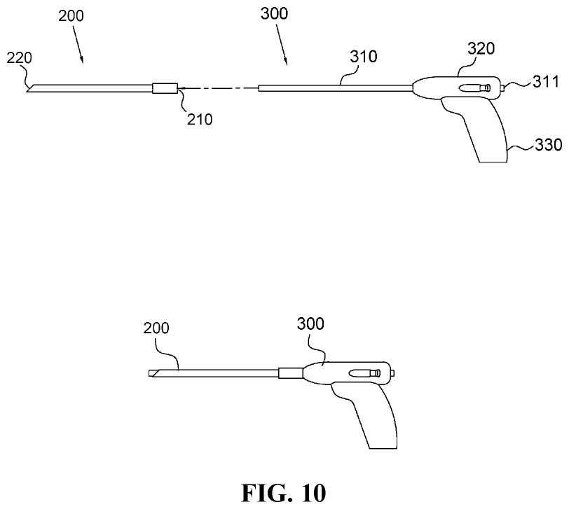

FIG. 10 is a perspective view illustrating joining of the intubator 200 and the endoscope 300 according to one embodiment of the present invention. The endoscope 300 is inserted into and joined with the intubator 200.

Referring to FIG. 10, the insertion portion 310 of the endoscope 300 is inserted into the intubator 200 through the fore end opening portion 210 provided on the other end of the intubator 200. Here, the insertion portion 310 of the endoscope 300 may have a length that protrudes a certain length through the distal end opening portion 220 formed on the one end of the intubator 200.

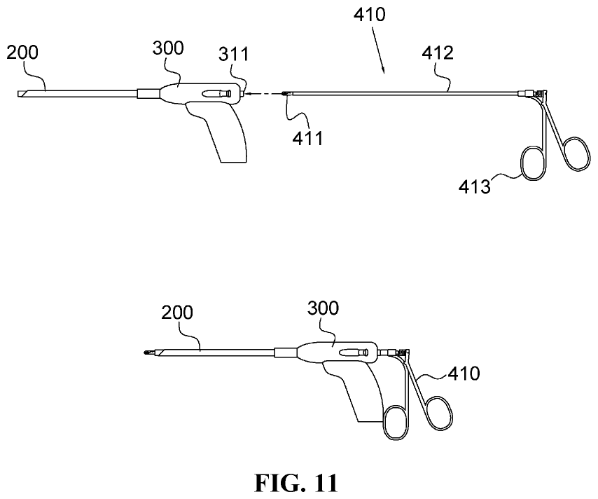

FIGS. 11 to 13 are perspective views illustrating joining of the intubator 200, the endoscope 300, and the surgical instrument 400 according to one embodiment of the present invention. The intubator 200 and the endoscope 300 are joined with each other, and then the surgical instrument 400 is joined with the inside of the endoscope 300.

FIG. 11 is a view illustrating the forceps 410 being inserted into the endoscope 300 according to one embodiment of the present invention.

Referring to FIG. 11, the rod portion 412 of the forceps 410 is inserted into the guide hole 311 formed in the insertion portion 310 of the endoscope 300. Here, the pincer portion 411 provided on the one end of the rod portion 412 is joined to protrude from a distal end of the guide hole 311 of the endoscope 300. In detail, the rod portion 412 may have a length with which the pincer portion 411 provided on the one end of the rod portion 412 may pass through the guide hole 311 in the endoscope 300 and approach the thickened yellow ligament that is the surgery target part T.

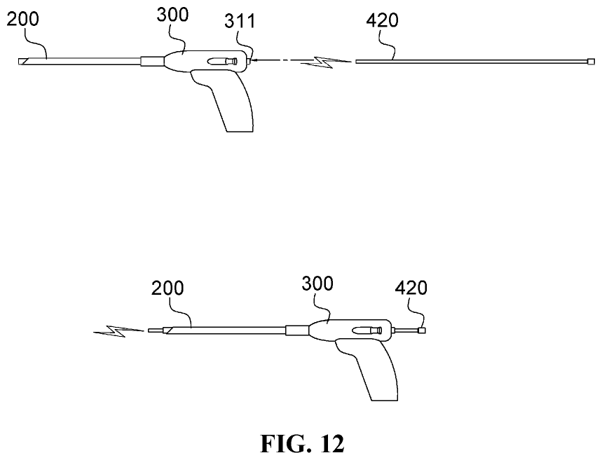

FIG. 12 is perspective views illustrating joining of the intubator 200, the endoscope 300, and the ultrasonic instrument 420 according to one embodiment of the present invention.

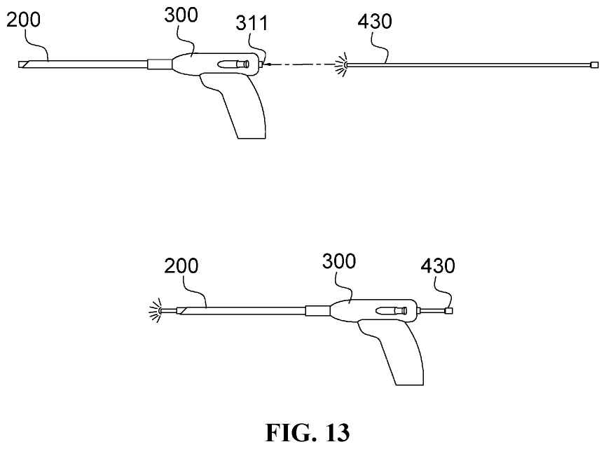

FIG. 13 is perspective views illustrating joining of the intubator 200, the endoscope 300, and the laser instrument 430 according to one embodiment of the present invention.

As shown in FIGS. 12 and 13, like the forceps 410, the ultrasonic instrument 420 or the laser instrument 430 is joined so that an ultrasonic device or a laser device passes through the guide hole 311 formed in the endoscope 300 and protrudes from the distal end of the one end of the guide hole 311.

In detail, the ultrasonic instrument 420 and the laser instrument 430 necessarily have lengths with which the ultrasonic device or laser device provided on one end of the ultrasonic instrument 420 or the laser instrument 430 may protrude through the distal end of the one end portion of the guide hole 311 of the endoscope 300 and may approach the thickened yellow ligament that is the surgery target part T while the other end is outside the body portion 320 of the endoscope 300.

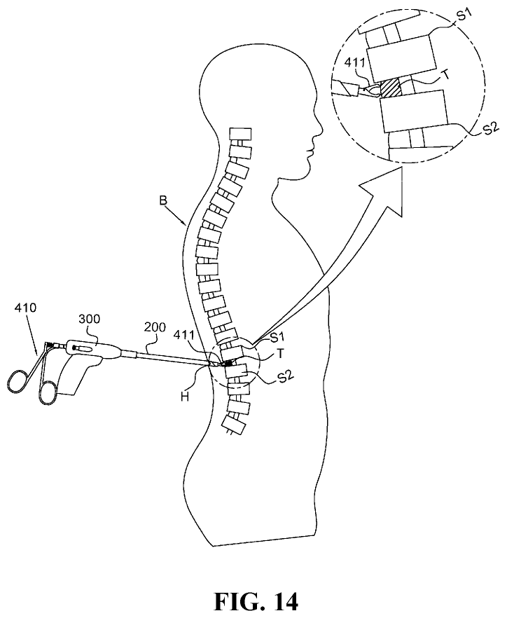

Hereinafter, one embodiment of a surgical operation according to the present invention will be described with reference to FIG. 14.

FIG. 14 is a perspective view illustrating a use state of a surgical instrument set according to the present invention. The surgical instrument set according to one embodiment of the present invention is inserted into the skin of a patient B through the treatment hole H formed by the punch 100 and approaches the surgery target part T to perform surgery. In detail, the thickened yellow ligament that is the surgery target part T is a connection structure which connects upper and lower laminas of a vertebral arch located behind a first vertebra S1 and a second vertebra S2 and will be removed when pressing nerves of the dura or the like due to hypertrophy and calcification caused by degenerative changes.

Referring to FIG. 14, the treatment hole H is formed at a position at which a shortest distance for the punch 100 to reach the surgery target part T from the skin or a path for minimizing injury to internal organs can be secured. In detail, the treatment hole H may be formed from the skin of the back located on an upper side between the first vertebra S1 and the second vertebra S2 in which the thickened yellow ligament that is the surgery target part T is located to perform invasion, but is not limited thereto and may be formed from skin on the side, abdomen, or the like according to a position of the surgery target part T to perform invasion. Subsequently, as one embodiment of the present invention, a surgical instrument set formed of a combination of the intubator 200, the endoscope 300, and the forceps 410 is inserted into the skin of a diseased area of the patient B through the formed treatment hole H and approaches the thickened yellow ligament that is the surgery target part T. Here, according to the purpose and target of surgery, the forceps 410 may be replaced with the ultrasonic instrument 420, the laser instrument 430, or another appropriate surgical instrument.

In detail, the forceps 410 or the ultrasonic instrument 420 grips or cuts the thickened yellow ligament that is the surgery target part T and then is released from the endoscope 300, and then the laser instrument 430 is inserted into the endoscope 300 to suture a gripped or cut part of the thickened yellow ligament that is the surgery target part T.

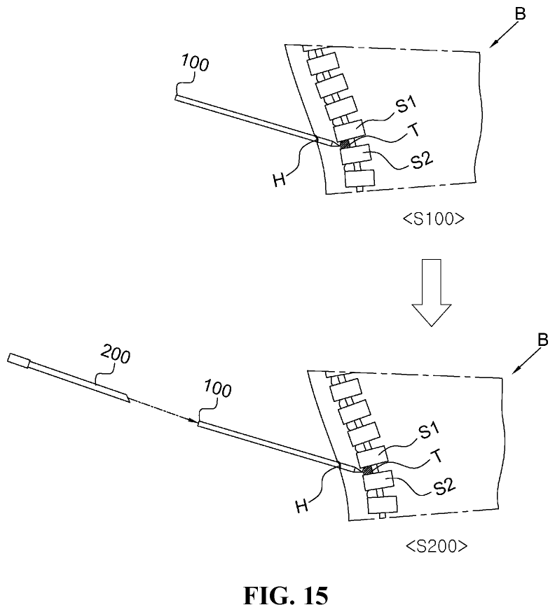

Hereinafter, an operating of joining the respective components of the present invention with one another will be described with reference to FIGS. 15 to 18. The present invention provides a joining operation in which the punch 100 forms and is inserted into the treatment hole H in the skin above the surgery target part T, the intubator 200 is inserted along the outer circumferential surface of the punch 100, the punch 100 is separated from the intubator 200, the endoscope 300 is inserted into the intubator 200, and the surgical instrument 400 is inserted into the endoscope 300.

FIG. 15 is a flowchart illustrating an operation of joining the punch 100 with the intubator 200 (S100) according to one embodiment of the present invention. In the operation, the intubator 200 is joined along the outer circumferential surface of the punch 100.

Referring to FIG. 15, <S100> is an operation of forming, by the punch 100, the treatment hole H in the skin on the surgery target part T. In detail, the punch 100 forms the treatment hole H by invading the skin on the thickened yellow ligament that is the surgery target part T and enters the skin of the patient B. Here, the treatment hole H may be formed at a position at which a shortest distance for the punch 100 to reach the surgery target part T from the skin or a path for minimizing injury to internal organs can be secured.

Referring to FIG. 15, <S200> is an operation of joining the intubator 200 along the punch 100. In detail, the other end of the punch 100 is inserted into the distal end opening portion 220 provided on the one end of the intubator 200, and thus the intubator 200 is joined along the outer surface of the punch 100.

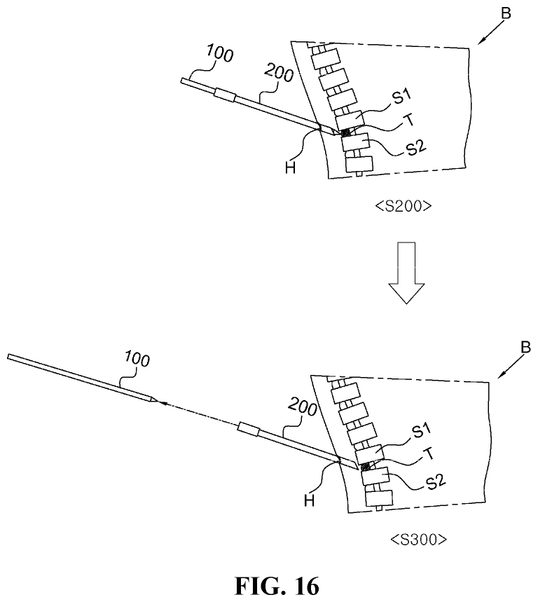

FIG. 16 is a flowchart illustrating an operation of separating the punch 100 from the intubator 200 (S300) according to one embodiment of the present invention. The intubator 200 is joined along the outer circumferential surface of the punch 100 and approaches the surgery target part T, and then the punch 100 is separated from the intubator 200.

Referring to FIG. 16, in <S200>, the intubator 200 is joined along the outer circumferential surface of the punch 100, is inserted through the treatment hole H, and enters a human body B. In detail, the intubator 200 is inserted to such a degree that the head portion 110 provided on the fore end of the punch 100 protrudes through the distal end opening portion 220 provided on the one end of the intubator 200. With this operation, a surgery space capable of allowing the surgical instrument 400 inserted into the intubator 200 through the endoscope 300 to adequately approach the thickened yellow ligament that is the surgery target part T is secured.

Referring to FIG. 16, in <S300>, the intubator 200 is inserted to a position close to the surgery target part T, and then the intubator 200 and the punch 100 are released from each other. In detail, the practitioner catches and takes the distal end of the other end portion of the punch 100 which protrudes from the other end of the intubator 200 so that the punch 100 is discharged from the skin of the patient B through the tub inside the intubator 200. Here, the intubator 200 maintains the treatment hole H.

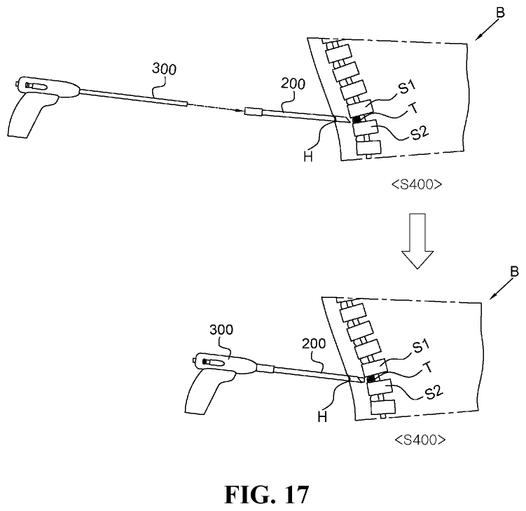

FIG. 17 is a flowchart view illustrating an operation of joining the intubator 200 with the endoscope 300 (S400) according to one embodiment of the present invention. After the punch 100 is removed from the inside of the intubator 200, the endoscope 300 is inserted into the intubator 200 inserted to the surgery target part T.

Referring to FIG. 17, in <S400>, after the joining of the punch 100 and the intubator 200 is released, the endoscope 300 enters the intubator 200. In detail, one end of the endoscope 300 is inserted into the intubator 200 through the other end of the intubator 200. In detail, the endoscope 300 is inserted into the intubator 200 and thus is joined to approach the thickened yellow ligament that is the surgery target part T.

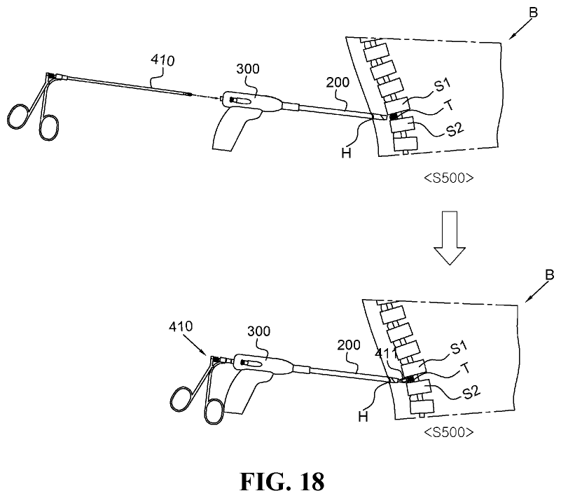

FIG. 18 is a flowchart view illustrating an operation of joining the endoscope 300 with the forceps 410 (S500) according to one embodiment of the present invention. The forceps 410 that is one embodiment of the surgical instrument 400 is inserted into the endoscope 300 inserted into the intubator 200.

Referring to FIG. 18, in <S500>, after the joining of the intubator 200 and the endoscope 300 is finished, the forceps 410 that is one embodiment of the surgical instrument 400 enters the endoscope 300. In detail, one end of the forceps 410 enters the endoscope 300 through the other end. Here, according to the purpose and target of surgery, the forceps 410 may be replaced with the ultrasonic instrument 420, the laser instrument 430, or another appropriate surgical instrument.

In more detail, the forceps 410 is inserted into the insertion portion 310 of the endoscope 300 and approaches the surgery target part T. Here, the forceps 410 may be inserted to such a degree that it protrudes through the distal end of the one end portion of the endoscope 300 to allow a distal end of the forceps 410 to approach the thickened yellow ligament that is the surgery target part T. The surgical instrument set joined through the above process approaches the thickened yellow ligament that is the surgery target part T and performs treatment such as gripping, cutting, suturing, and the like according to the purpose of surgery.

Meanwhile, to determine whether the forceps 410 has appropriately reached the surgery target part T before performing surgery using the forceps 410, preferentially, the practitioner removes blood near the surgery target part T through the abstergent supply portion 312 and the abstergent discharge portion 314 of the endoscope 300, takes images using the image-capturing portion 313, and sees, with the naked eye, whether the forceps 410 is appropriately located on the surgery target part T. Subsequently, when the surgery target part T is treated using the forceps 410, as described above, the treatment may be performed while monitoring a state of the surgery target part T using the abstergent supply portion 312, the abstergent discharge portion 314, and the image-capturing portion 313.

The above exemplary embodiments of the present invention have been disclosed for the purpose of exemplification, and it should be noted that a variety of modifications, changes, and additions can be made by those of ordinary skill in the art without departing from the concept and scope of the present invention and the modifications, changes, and additions are included in the scope of the claims. Also, the surgical target part T of the present invention is not limited to the thickened yellow ligament.

Since a variety of substitutions, modifications, and changes may be made by those of ordinary skill in the art without departing from the technical concept of the present invention, the present invention is not limited by the above embodiments or the attached drawings.

Although the methods used in the above-described exemplary system have been described as a series of operations or blocks on the basis of the flowcharts, the present invention is not limited to this order of operations, and any one of the operations may be performed in a different order with another operation or at the same time. Also, it can be understood by those of ordinary skill in the art that the operations shown in the flowcharts are not exclusive, and other operations may be included or one or more operations can be deleted without influencing the scope of the present invention.

1. A surgical instrument set for single port spinal surgery, comprising:
a punch (100) configured to form a treatment hole (H) in the skin above a surgery target part (T);
an intubator (200) having a tubular shape and inserted into the treatment hole (H) according to the punch (100) to secure a surgery space;
an endoscope (300) comprising an insertion portion (310) having a rod shape to be inserted into the intubator (200), in which a guide hole (311) having a tubular shape is opened, and comprising an image-capturing portion (313) configured to take images of the surgery target part (T); and
a surgical instrument (400) inserted into the guide hole (311) to perform surgery on the surgery target part (T).

2. The surgical instrument set of claim 1, wherein the insertion portion (310) comprises:
an abstergent supply portion (312) configured to supply an abstergent to one end of the insertion portion (310); and
an abstergent discharge portion (314) configured to discharge the supplied abstergent.

3. The surgical instrument set of claim 1, wherein the surgical instrument (400) is a forceps (410),
wherein the forceps (410) comprises:
a rod portion (412) inserted into the guide hole (311);
a pincer portion (411) provided on one end of the rod portion (412) and configured to grip the surgery target part (T); and
a manipulation portion (413) provided on the other end of the rod portion (412) and configured to manipulate the pincer portion (411), and
wherein only the rod portion (412) and the pincer portion (411) are inserted into the guide hole (311).

4. The surgical instrument set of claim 3, wherein the rod portion (412) has a tubular shape,
wherein the pincer portion (411) comprises a second pincer (4112) fixed to one end of the rod portion (412) and a first pincer (4111) hinge-connected to the one end of the rod portion (412),
wherein the rod portion (412) further comprises a transfer portion (412a) therein which has one end connected to the first pincer (4111) and the other end connected to the manipulation portion (413), and
wherein the transfer portion (412a) is moved forward or backward by manipulating the manipulation portion (413) so that the first pincer (4111) moves like pincers.

5. The surgical instrument set of claim 1, wherein the intubator (200) has one cross section including an inclined surface.

6. The surgical instrument set of claim 1, wherein the surgical instrument (400) is at least one of a forceps (410), an ultrasonic instrument (420), and a laser instrument (430).

7. A method of using a surgical instrument set for single port spinal surgery, comprising:
an operation (S100) of forming a treatment hole (H) in the skin above a surgery target part (T) using a punch (100);
an operation (S200) of securing a surgery space by inserting an intubator (200) having a tubular shape into the treatment hole (H) along the punch (100);
an operation (S300) of removing the punch (100) from the intubator (200);
an operation (S400) of inserting an endoscope (300) including an insertion portion (310) in which a guide hole (311) is formed and which comprises an image-capturing portion (313) into the intubator (200); and
an operation (S500) of performing surgery on the surgery target part (T) by inserting the surgical instrument (400) into the guide hole (311).

8. The method of claim 7, wherein at least one of a forceps (410) configured to grip or remove the surgery target part (T), an ultrasonic instrument (420), and a laser instrument (430) is used in the operation (S500) of performing of the surgery on the surgery target part (T) by inserting the surgical instrument (400) into the guide hole (311).

9. The method of claim 7, wherein, after the operation (S400) of inserting the endoscope (300) including the insertion portion (310) in which the guide hole (311) is formed and which comprises the image-capturing portion (313) into the intubator (200), a view of the image-capturing portion (313) is secured by cleaning the surgery target part (T) using an abstergent supply portion (312) and an abstergent discharge portion (314) which are provided in the insertion portion (310) to supply or suction an abstergent.
